(19) United States
(12) Patent Application Publication
(10) Pub. No.: US 2021/0364778 A1
(43) Pub. Date: Nov. 25, 2021

(54) ENDOSCOPE DEVICE

(71) Applicant: ALTEK BIOTECHNOLOGY CORPORATION, Hsinchu City (TW)

(72) Inventors: Hsi-Hsin Loo, Taipei City (TW); Chun-Wei Liu, Hsinchu County (TW); Liang-Yi Li, Hsinchu County (TW)

(73) Assignee: ALTEK BIOTECHNOLOGY CORPORATION, Hsinchu City (TW)

(21) Appl. No.: 16/920,741

(22) Filed: Jul. 5, 2020

(30) Foreign Application Priority Data
May 22, 2020 (TW) 109206307

(51) Int. Cl. G02B 23/24; A61B 1/00; A61B 1/06; A61B 1/05

(52) U.S. Cl. CPC G02B 23/243; G02B 23/2484; A61B 1/05; A61B 1/00195; A61B 1/0661; G02B 23/2461

(57) ABSTRACT

An endoscope device includes an insertion portion. The insertion portion includes a distal end. The distal end includes a lens holder, a lens disposed in the lens holder, an image sensor corresponding to the lens and a flexible circuit board electrically connected to the image sensor and a circuit board holder. The flexible circuit board is jointly clamped by the circuit board holder and the lens holder.

### CROSS-REFERENCE TO RELATED APPLICATION

This application claims the priority benefit of Taiwan application no. 109206307, filed on May 22, 2020. The entirety of the above-mentioned patent application is hereby incorporated by reference herein and made a part of this specification.

### BACKGROUND

### Technical Field

This disclosure is related to an endoscope device, and in particular to an insertion portion of an endoscope device.

### Description of Related Art

With the technological advancement of semiconductors and micromachines, the application of video endoscopes has popularized gradually. Compared to the complicated optical system in traditional optical endoscopes, the simplified structure of video endoscopes further miniaturizes the endoscope, resulting in not only the reduction of the size of the endoscope, but also the improvement in the resolution and image quality, and making the shift of the image sensor and illuminating device towards the distal end of the endoscope a systemic development trend.

With the reduction in the diameter of the endoscope and the concentration of the electronic components, image sensor and related parts at the distal end of the endoscope, the stacking density of the components located at the distal end of the insertion portion of the endoscope has increased, making it more difficult to position the related components. In the pursuit of good imaging quality, the optimization of the positioning of these components at the distal end of the endoscope is vital.

### SUMMARY

This disclosure provides an endoscope device whose elements at the distal end of the insertion portion are well positioned.

An embodiment of the disclosure is an endoscope device, including the insertion portion. The insertion portion has a distal end, and the distal end includes a lens holder, a lens disposed in the lens holder, an image sensor corresponding to the lens, a flexible circuit board electrically connected to the image sensor and a circuit board holder, wherein the circuit board holder and the lens holder jointly clamp the flexible circuit board.

In an embodiment of the disclosure, the above endoscope device further includes a handle part. The handle part is connected to the insertion portion.

In an embodiment of the disclosure, the abovementioned distal end further includes a light source, which is electrically connected to the flexible circuit board and leans against the lens holder.

In an embodiment of the disclosure, the abovementioned distal end further includes a light guiding housing, and the light guiding housing includes an optical element, and the optical element is positioned in front of the light source.

In an embodiment of the disclosure, the abovementioned optical element includes a convex lens.

In an embodiment of the disclosure, the abovementioned light guiding housing includes a polygonal hole, and the lens holder is sleeved in the polygonal hole of the light guiding housing.

In an embodiment of the disclosure, the abovementioned distal end further includes an optical lens and a waterproof light-shielding adhesive layer, wherein the optical lens is located in front of the lens, and the waterproof light-shielding adhesive layer is provided next to the periphery of the optical lens to fix the optical lens to the lens holder and to block and prevent unexpected stray light originating from the light source, the light guiding column and the external environment from entering the lens and affecting the image quality.

In an embodiment of the disclosure, the abovementioned flexible circuit board includes a first segment, a second segment bent to be connected to the first segment, a third segment and a fourth segment bent to be connected to two sides of the second segment, a fifth segment bent to be connected to the fourth segment and a sixth segment bent to be connected to the third segment, wherein the first segment extends away from the distal end, and the second segment, the third segment and the sixth segment surround a part of the lens holder.

In an embodiment of the disclosure, the abovementioned circuit board holder and the lens holder jointly clamp the fifth segment, and the image sensor is disposed at the fifth segment.

In an embodiment of the disclosure, the abovementioned distal end further includes a light source disposed at the sixth segment, and the sixth segment is adhered to the lens holder.

In an embodiment of the disclosure, either of the abovementioned circuit board holder or the lens holder includes a fixing protruding part, while the other includes a fixing groove. The fixing protruding part extends into the fixing groove, and the fixing protruding part extends in the direction that is parallel to the extension direction of the insertion portion.

In summary, the insertion portion of the endoscope device in the disclosure fixes the lens by the lens holder, and clamps of the flexible circuit board jointly by the circuit board holder and the lens holder to fix the relative positions of the internal components at the distal end, such that the internal components at the distal end of the insertion portion can be well positioned.

### BRIEF DESCRIPTION OF THE DRAWINGS

FIG. 1 is a schematic diagram of an endoscope device according to an embodiment of the disclosure.

FIG. 2 is a schematic cross-sectional view of the distal end of the endoscope device in FIG. 1.

FIG. 3A is a schematic stereoscopic view of a light guiding housing of the distal end in FIG. 2.

FIG. 3B is a schematic perspective view of a light guiding housing according to an embodiment of the disclosure.

FIG. 4 is a schematic perspective view of a housing that conceals the distal end of the endoscope device in FIG. 1.

FIG. 5 is a schematic diagram showing the lens and the image sensor in FIG. 4 after being cut.

FIG. 6 is a partial cross-sectional schematic diagram of FIG. 1 taken along the line A-A.

FIG. 7 is a partial cross-sectional schematic diagram of FIG. 1 taken along the line B-B.

FIG. 8 is a partial cross-sectional schematic diagram of an endoscope device according to another embodiment of the disclosure.

### DESCRIPTION OF THE EMBODIMENTS

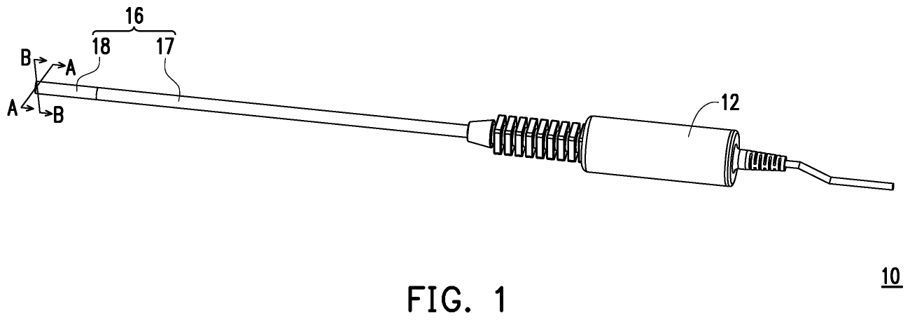

FIG. 1 is a schematic diagram of an endoscope device according to an embodiment of the disclosure. Referring to FIG. 1, the endoscope device 10 of the embodiment can be applied to video endoscopes, but is not limited thereto. The endoscope device 10 includes a handle part 12 and an insertion portion 16. The handle part 12 is for the operator to hold on to. The handle part 12 is connected to the insertion portion 16. The insertion portion 16 includes an extension portion 17, which is connected to the handle part 12, and a distal end 18 that is away from the handle part 12.

Note that the handle part 12 and the insertion portion 16 may be formed integrally. Alternatively, the insertion portion 16 may be detachable from the handle part 12 for single use or serves as a replaceable structure. The disclosure provides no limitation to the connection relationship between the handle part 12 and the insertion portion 16. The emphasis in this disclosure lies in the internal structure of insertion portion 16, which is described in detail below.

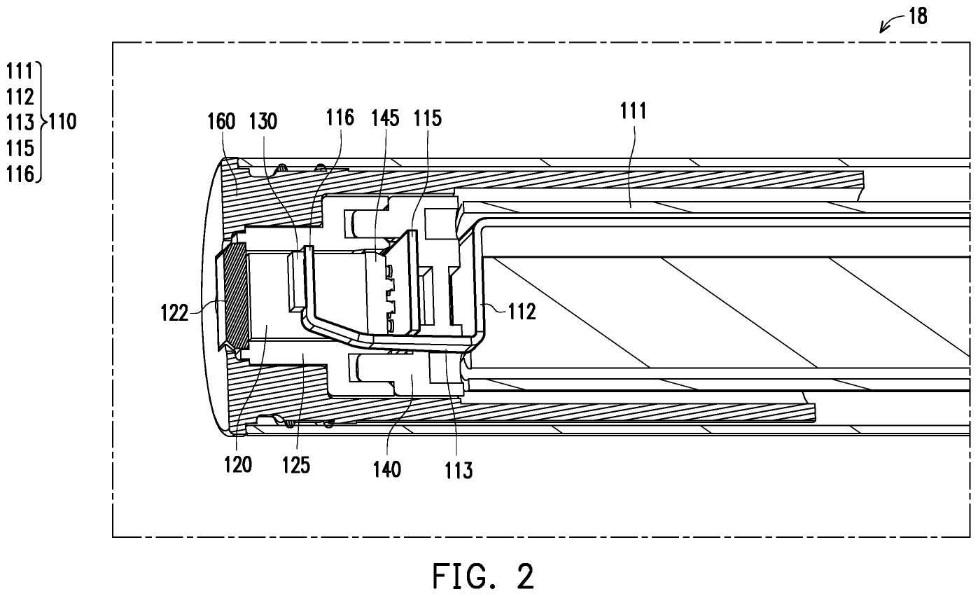

FIG. 2 is a schematic cross-sectional view of the distal end of the endoscope device in FIG. 1. Referring to FIG. 2, the distal end 18 of the insertion portion 16 (see FIG. 1) includes a lens holder 125, a lens 120 disposed in the lens holder 125, an image sensor 145 corresponding to the lens 120, a flexible circuit board 110 electrically connected to the image sensor 145 and a circuit board holder 140.

The lens 120 in the distal end 18 is fixed by the lens holder 125. The image sensor 145 is disposed on the flexible circuit board 110, and the circuit board holder 140 and the lens holder 125 jointly clamp the flexible circuit board 110 to fix the flexible circuit board 110, so that the lens 120, the image sensor 145, and the flexible circuit board 110 are well positioned within the distal end 18.

In addition, the distal end 18 also includes an optical lens 122, which is located in front of the lens 120, and the optical lens 122 is fixed to the lens holder 125. In other words, the lens holder 125 is made of a dark opaque material, and may be configured to fix the relative positions of the optical lens 122 and lens 120, and also blocks the stray light originating from the light source.

In the embodiment, the insertion portion 16 further includes a light source 130, which is electrically connected to the flexible circuit board 110, and the light source 130 is disposed at the distal end 18 and located beside the lens 120. In the embodiment, the light source 130 leans against the lens holder 125.

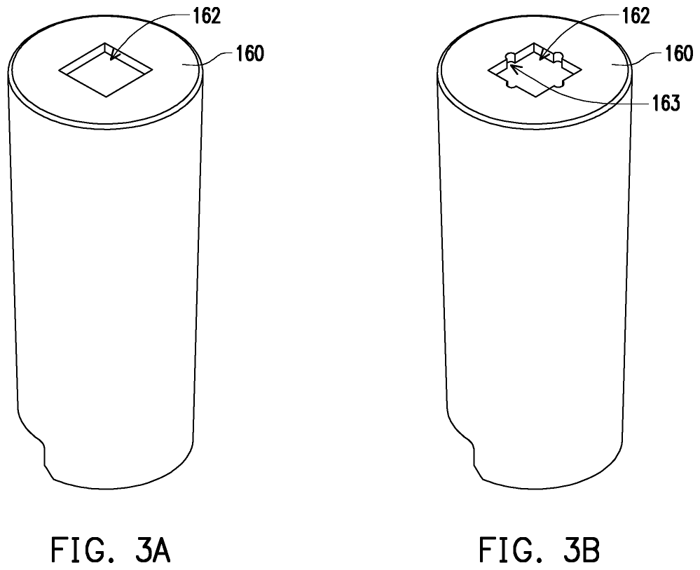

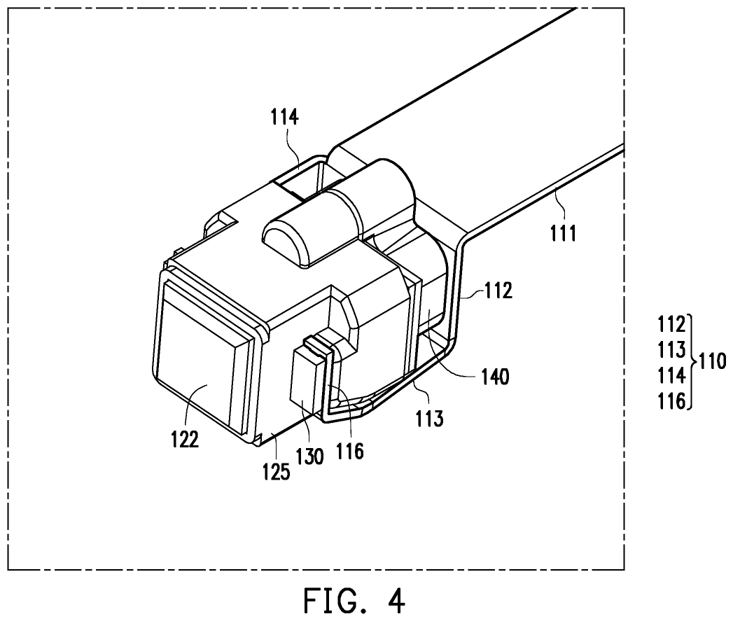

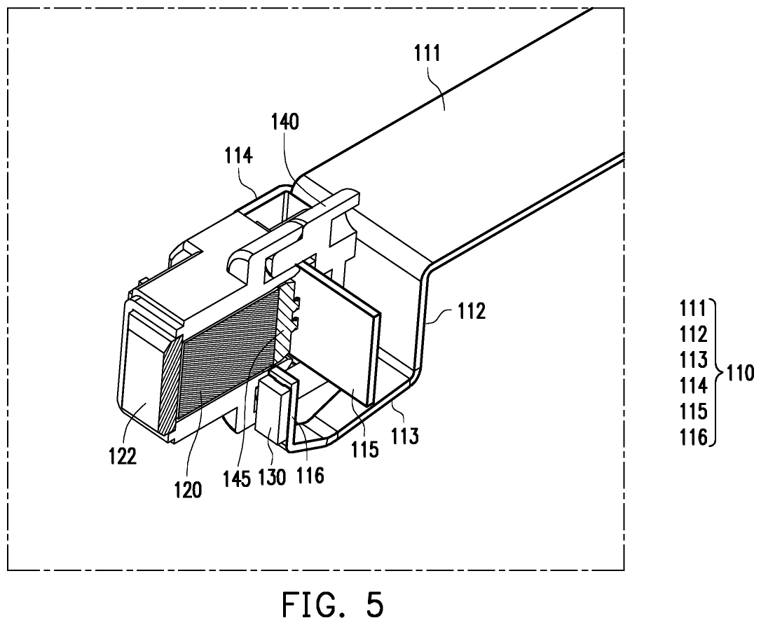

FIG. 3A is a schematic perspective view of a light guiding housing of the distal end in FIG. 2. FIG. 3B is a schematic perspective view of a light guiding housing according to an embodiment of the disclosure. FIG. 4 is a schematic perspective view of a housing that conceals the distal end of the endoscope device in FIG. 1. FIG. 5 is a schematic diagram showing the lens and the image sensor in FIG. 4 after being cut.

Referring to FIG. 2 to FIG. 4, the distal end 18 further includes a light guiding housing 160. As shown in FIG. 3A, the light guiding housing 160 has a polygonal hole 162, and the lens holder 125 is sleeved in the polygonal hole 162 of the light guiding housing 160. The outer contour of the lens holder 125 conforms to the contour of the polygonal hole 162. For example, the polygonal hole 162 is shaped as a square, but the shape of the polygonal hole 162 is not limited thereto. As shown in FIG. 3B, one or more small notches 163, for example, may be added next to the polygonal hole 162 to facilitate the dispensing process. The design of the polygonal hole 162 not only prevents the lens holder 125 from rotating relative to the light guiding housing 160, but also ensures that the lens holder 125 is well positioned within the light guiding housing 160.

In addition, the flexible circuit board 110 includes a first segment 111, a second segment 112 bent to be connected to the first segment 111, a third segment 113 and a fourth segment 114 bent to be connected to two sides of the second segment 112, a fifth segment 115 (see FIG. 5) bent to be connected to the fourth segment 114 and a sixth segment 116 bent to be connected to the third segment 113, wherein the first segment 111 extends away from the distal end 18, and the second segment 112, the third segment 113 and the sixth segment 116 surround a part of the lens holder 125. The sixth segment 116 leans against the lens holder 125 and located next to the lens 120. In the embodiment, the light source 130 is disposed on the sixth segment 116, and the sixth segment 116 is adhered to the lens holder 125 to fix the position of the light source 130.

In the embodiment, the material of the lens holder 125 includes plastic, thermally conductive plastic or metal, and the material of the circuit board holder 140 includes plastic or any material with good thermal conductivity to facilitate heat dissipation, but the disclosure provides no limitation to the material of the lens holder 125 and circuit board holder 140.

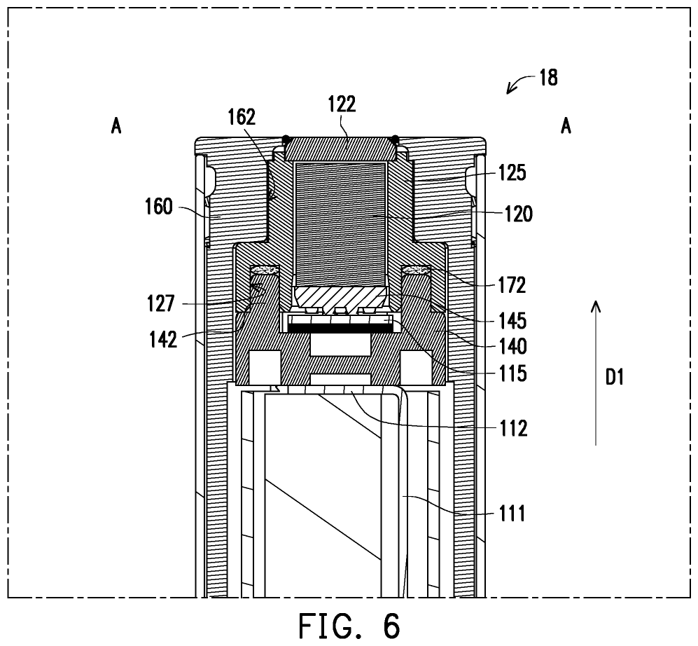

FIG. 6 is a partial cross-sectional schematic diagram of FIG. 1 taken along the line A-A. Referring to FIG. 6, in the embodiment, either of the circuit board holder 140 or the lens holder 125 includes a fixing protruding part 142, while the other includes a fixing groove 127, and the fixing protruding part 142 extends into the fixing groove 127. With this structure, the circuit board holder 140 and the lens holder 125 can be well positioned onto each other. In the embodiment, both the fixing protruding part 142 and the insertion portion 16 extend along the extension direction Dl.

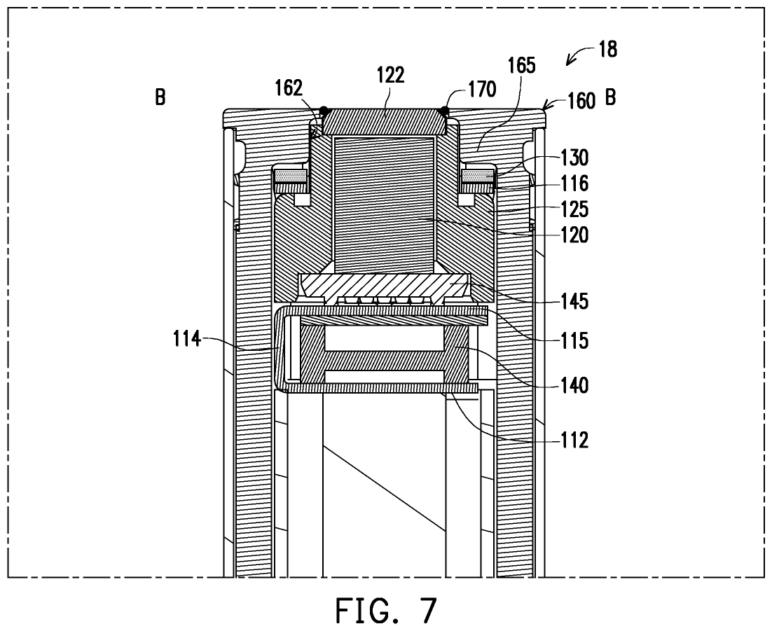

FIG. 7 is a partial cross-sectional schematic diagram of FIG. 1 taken along the line B-B. Referring to FIG. 7, in the embodiment, the distal end 18 further includes a waterproof light-shielding adhesive layer 170, and the waterproof light-shielding adhesive layer 170 is provided at the periphery of the optical lens 122 to fix the optical lens 122 to the lens holder 125. The waterproof light-shielding adhesive layer 170 not only blocks water and moisture in the air to prevent water and moisture in the air from entering the interior of the distal end 18 but also blocks unexpected light, so as to prevent the low-angle stray light originating from the light source 130 and the light guiding housing 160 from entering the optical lens 122 and adversely affecting the image.

With the above configuration, the optical lens 122, the lens 120, the image sensor 145, the flexible circuit board 110 and the light source 130 are well fixed in position through the circuit board holder 140 and the lens holder 125, and the abovementioned components are closely arranged in the distant end 18 with a small diameter.

In addition, in the embodiment, in order to provide a specific illuminating effect, the light guiding housing 160 includes an optical element 165, which is located in front of the light source 130. For example, the optical element 165 produces a desired optical effect by having a specific thickness or shape. Alternatively, the optical element 165 may be doped with diffusion particles or phosphor powder, and in an embodiment, the optical element 165 may also produce a uniform light effect.

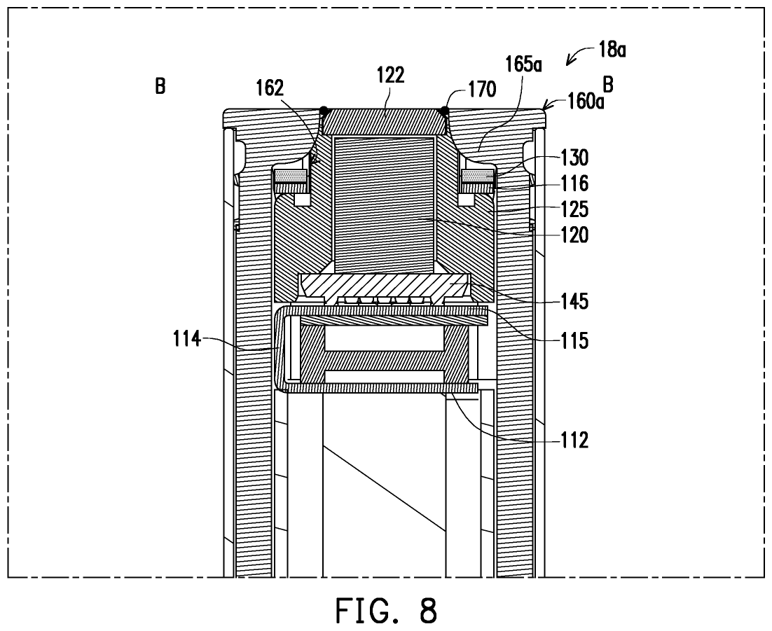

FIG. 8 is a partial cross-sectional schematic diagram of an endoscope device according to another embodiment of the disclosure. Referring to FIG. 8, in the distal end 18a of the embodiment, the optical element 165a of the light guiding housing 160a includes a convex lens to converge the light to a specific position. Note that the types and shapes of the optical elements 165 and 165a are not limited thereto.

In summary, the endoscope device in the disclosure fixes the lens by the lens holder, and clamps of the flexible circuit board jointly by the circuit board holder and the lens holder to fix the relative positions of the internal components at the distal end, such that the internal components at the distal end can be well positioned.

What is claimed is:

1. An endoscope device, comprising:
an insertion portion with a distal end comprising a lens holder, a lens disposed in the lens holder, an image sensor corresponding to the lens, a flexible circuit board electrically connected to the image sensor and a circuit board holder, wherein the circuit board holder and the lens holder jointly clamp the flexible circuit board.

2. The endoscope device according to claim 1, further comprising:
a handle part connecting to the insertion portion.

3. The endoscope device according to claim 1, wherein the distal end further comprising a light source electrically connected to the flexible circuit board, and the light source leans against the lens holder.

4. The endoscope device according to claim 3, wherein the distal end further comprises a light guiding housing, the light guiding housing comprises an optical element, and the optical element is positioned in front of the light source.

5. The endoscope device according to claim 4, wherein the optical element comprising a convex lens.

6. The endoscope device according to claim 4, wherein the light guiding housing has a polygonal hole, and the lens holder is sleeved in the polygonal hole of the light guiding housing.

7. The endoscope device according to claim 1, wherein the distal end further comprises an optical lens and a waterproof light-shielding adhesive layer, the optical lens is located in front of the lens, and the waterproof light-shielding adhesive layer is provided next to a periphery of the optical lens to fix the optical lens to the lens holder.

8. The endoscope device according to claim 1, wherein the flexible circuit board comprises a first segment, a second segment bent to be connected to the first segment, a third segment and a fourth segment bent to be connected to two sides of the second segment, a fifth segment bent to be connected to the fourth segment and a sixth segment bent to be connected to the third segment, wherein the first segment extends away from the distal end, and the second segment, the third segment and the sixth segment surround a part of the lens holder.

9. The endoscope device according to claim 8, wherein the circuit board holder and the lens holder jointly clamp the fifth segment, and the image sensor is disposed at the fifth segment.

10. The endoscope device according to claim 8, wherein the distal end further comprises a light source disposed at the sixth segment, and the sixth segment is adhered to the lens holder.

11. The endoscope device according to claim 1, wherein either of the circuit board holder or the lens holder comprises a fixing protruding part, while the other comprises a fixing groove, the fixing protruding part extends into the fixing groove, and the fixing protruding part extends in a direction that is parallel to an extension direction of the insertion portion.
